(19) United States
(12) Patent Application Publication
(10) Pub. No.: US 2025/0256121 A1
(43) Pub. Date: Aug. 14, 2025

(54) Therapeutic Animal Collar

(71) Applicant: Erchonia Corporation, LLC, Fountain Inn, SC (US)

(72) Inventors: Travis Sammons, Fountain Inn, SC (US); Steven C Shanks, Fountain Inn, SC (US)

(73) Assignee: Erchonia Corporation, LLC, Fountain Inn, SC (US)

(21) Appl. No.: 19/038,973

(22) Filed: Jan. 28, 2025

Related U.S. Application Data
(60) Provisional application No. 63/551,267, filed on Feb. 8, 2024.

(51) Int. Cl. A61N 5/06

(52) U.S. Cl. CPC A61N 5/0613; A61N 2005/0645; A61N 2005/0663; A61N 2005/0666

(57) ABSTRACT

A collar worn around the neck of an animal delivers non-invasive light energy to free-floating mitochondria flowing through the animal's veins and arteries. The collar comprises a housing which holds at least one light source, a power source, a controller and in some configurations a reflector. The housing has an aperture through which the light energy is emitted towards the animal's tissue. The housing is connected to a band or harness, which positions the housing where the neck broadens out to the shoulder. Preferably the collar is secured around the neck of the animal loosely enough that the housing falls naturally by gravity to where the neck broadens out to the shoulder. The non-invasive light energy impinges the animal's fur or tissue and penetrates to free-floating mitochondria in the blood flowing through the animal's veins and arteries. This results in improved oxidative phosphorylation and mitochondrial function in the treated mitochondria.

### FIELD OF INVENTION

This invention relates generally to light-emitting therapeutic devices for animals. This invention relates specifically to a collar that emits light energy towards the neck of an animal to deliver therapeutic phototherapy for maintaining health and curing diseases.

### BACKGROUND

Veins contain free-floating mitochondria with an estimated number ranging from 200,000 to 3.7 million per milliliter of plasma. Free-floating mitochondria circulates through the entire body where they are taken up by injured cells, replenishing their depleted mitochondrial pool and enhancing their energy production capacity for repair. Plasma's constant circulation is crucial for maintaining the body's internal environment.

Phototherapy has been shown through numerous clinical studies and regulatory clearances to be a safe and effective, simple, non-invasive and side-effect free alternative to medication and surgical procedures for the reduction of symptoms in a variety of conditions. Light therapy, particularly noninvasive low-level laser therapy, reduces edema, improves wound healing, and relieves pain of various etiologies. It is also used in the treatment and repair of injured muscles and tendons. Phototherapy has been shown to have the potential to alter cellular metabolism to produce a beneficial clinical effect.

Based on its ability to modulate cellular metabolism and alter the transcription factors responsible for gene expression, phototherapy has been found to alter gene expression, cellular proliferation, intra-cellular pH balance, mitochondrial membrane potential, generation of transient reactive oxygen species and calcium ion level, proton gradient and consumption of oxygen. Stimulation of the mitochondria via low-energy light has been shown to provoke a dynamic shift in the function of an individual cell. Laser therapy has been shown to stimulate cell regeneration and later gene expression.

Light therapy is a commonly used procedure to provide therapeutic benefits across a wide range of conditions and diseases that affect humans and non-human animals. Currently the industry applies phototherapy to only the damaged or diseased area or tissue. This requires close contact with the animal and animal's cooperation to remain still to avoiding interfering with the treatment by moving or rubbing the light-emitting device off the body. For human animals this is relatively easy to accomplish, but for non-human animals it can be extremely challenging. It would be desirable to have a device that permits the animal to be treated with light therapy while continuing normal activities such as walking, running, sleeping, and eating.

An animal may have more than one ailment or an ailment that affects multiple areas and systems of the body. It would be advantageous to treat the entire body to influence a systemic therapeutic effect to maintain health and cure diseases throughout an animal's body. To that end, it would be advantageous to apply light energy to free-floating mitochondria to increase mitochondrial membrane potential.

### SUMMARY OF THE INVENTION

A collar is worn around the neck of an animal to deliver non-invasive light energy to free-floating mitochondria flowing through the animal's veins and arteries. The collar comprises a housing which holds at least one light source, a power source, a controller and in some configurations a reflector. The housing has an aperture through which the light energy is emitted towards the animal's tissue. The housing is connected to a band or harness, which positions the housing in the area where the neck broadens out to the shoulder. Preferably the collar is secured around the neck of the animal loosely enough that the housing falls naturally by gravity to the area where the neck broadens out to the shoulder. The non-invasive light energy impinges the animal's fur or tissue and penetrates to free-floating mitochondria in the blood flowing through the animal's veins and arteries. This results in improved oxidative phosphorylation and mitochondrial function in the treated mitochondria.

In one embodiment the device emits a single color light, preferably visible or NIR wavelengths such as red, violet-blue or green. In another embodiment the device emits at least three colors of light. For example, Complexes I and II are treated with violet-blue (400-490 nm) light. Complex III is treated with green (495-570 nm) light. Complex IV is treated with red (620-700 nm) light. To achieve optimal oxidative phosphorylation all three colors are applied to the neck, simultaneously or non-simultaneously. The light energy can be applied using a single light source that can emit all three wavelengths, or multiple light sources that emit a subset of the desired wavelengths.

### BRIEF DESCRIPTION OF THE DRAWINGS

FIG. 1 illustrates a dog wearing a therapeutic collar of the present invention using a band, in which the housing is disposed on the side of the dog's neck.

FIG. 2 illustrates a dog wearing a therapeutic collar of the present invention using a band, in which the housing is disposed on the underside of the dog's neck.

FIG. 3 illustrates a dog wearing a therapeutic collar of the present invention using a harness, where the housing is disposed on the side of the dog's neck.

FIG. 4 illustrates a dog wearing a therapeutic collar of the present invention using a harness, where the housing is disposed on the underside of the dog's neck.

FIG. 5 is a schematic illustration of the housing and its internal components with a reflector.

FIG. 6 is a schematic illustration of the housing and its internal components without a reflector.

FIG. 7 illustrates a line of laser light emitted from the housing.

FIG. 8 is a top perspective view of the therapeutic collar, in which the housing is attached to a band.

FIG. 9 illustrates the electron transport chain and the application of colored light to specific complexes.

FIG. 10 illustrates three colors of light passing through a lens resulting in white light.

### DETAILED DESCRIPTION OF THE INVENTION

The present invention is particularly suited to treating animals with light therapy which can't or won't remain still for the duration of the therapy. It is particularly suited to non-human mammals such as pets and livestock, including but not limited to canines, felines, equines, bovines, and caprines.

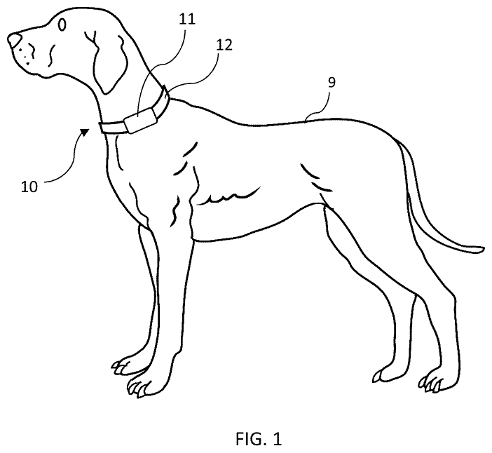

A collar 10 is worn around the neck of an animal 9 to deliver non-invasive light energy to the animal's veins and arteries which carry the free-floating mitochondria. See FIGS. 1-4. The collar 10 comprises a housing 11 which holds at least one light source 20, a power source 22, and a controller 23. The housing 11 has an aperture 14 through which the light energy is emitted towards the animal's tissue. The housing 11 is connected to a band 12 or harness 13, which positions the housing 11 to emit light towards the desired treatment area. In some configurations the housing 11 further comprises a reflector 21 to help aim the emitted light 17 through the aperture 14.

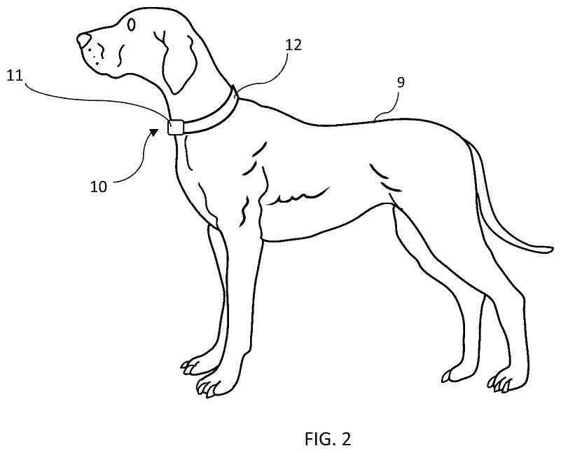

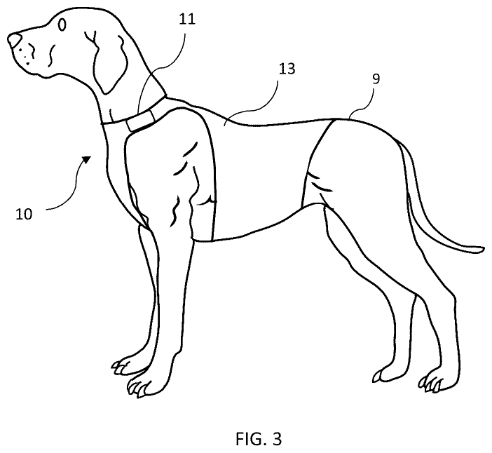

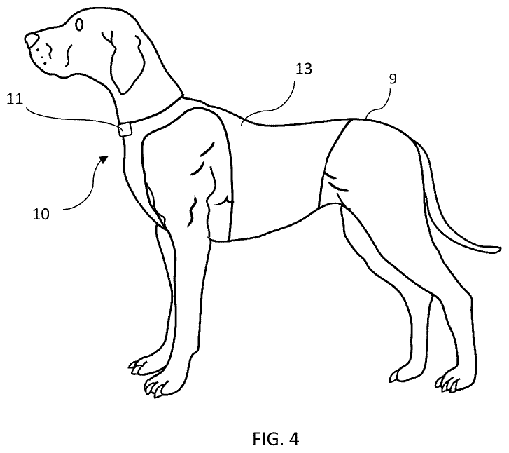

In a preferred embodiment, the housing 11 is positioned so that the emitted light impinges the tissue where the neck broadens out to the shoulder. Preferably the collar 10 is secured around the neck of the animal loosely enough that the housing falls naturally by gravity to the area where the neck broadens out to the shoulder. In this way the collar 10 seats itself in the desired location to treat veins and arteries during normal activities such as walking, running, sleeping, and eating, thereby minimizing the risk the device will shift away from the treatment area during treatment. In one embodiment, the collar treats oxygenated blood such as that found in the carotid artery so that the activity of free floating mitochondria in plasma is enhanced as it exits the heart and is distributed throughout the body. See FIGS. 1 and 3. In another embodiment the collar treats deoxygenated blood such as that found in the jugular or brachiocephalic veins so that the activity of free floating mitochondria in plasma is enhanced as it then enters the heart and is distributed throughout the body. See FIGS. 2 and 4. The light can penetrate fur to reach the tissue, either between the hairs or through the keratin of the fur.

FIGS. 1 and 3 show a dog 9 wearing a collar 10 in which the housing 11 is connected to a band 12. FIGS. 2 and 4 show a dog 9 wearing a collar 10 in which the housing 11 is connected to a harness 13. The band 12 or harness 13 may be leather, fabric, plastic, a metal chain, or other type of flexible material. The band 12 or harness 13 is preferably adjustable in length, although may come in standardized or fixed sizes for various animal species and breeds.

The housing 11 can be fixed to the band 12 or harness 13 or movably attached to it. The housing 11 can be fixed by permanent fasteners, such as being integral with or sewn to the band 12 or harness 13. The housing 11 can be movably attached to the band or harness 13 by, for example a slidable casing, Velcro®, or a row of snaps that permits the housing to be attached a variety of different points. The relative weights of the housing 11 and the collar fastener (and typically attached tags) can be balanced against one another to help ensure the housing 11 directs the therapeutic light to a desired area. For example, FIGS. 1 and 3 show the housing 11 fixed to the collar in a position to treat a dog's carotid artery, while FIGS. 2 and 4 show the housing 11 fixed to the collar in a position to treat the dog's jugular or brachiocephalic veins. The housing 11 can be weighted to fall towards the lowest point on the neck, so that the light traverses a pathway to arrive at and penetrate the animal's neck tissue proximate a vein or artery, thereby increasing mitochondrial membrane potential in the mitochondria in the vein or artery.

The light 17 can be from any light source 20 including lasers, light emitting diodes (LEDs), lamp, or the like. In a preferred embodiment the light source is a semiconductor laser diode. The light source needs to be small enough and lightweight enough to fit in the housing without overwhelming the size and strength of the animal's neck. The housing can be made less bulky if the light source lies parallel to the band as opposed to perpendicular to it. In some configurations the light source thus emits light parallel to the band 12. In such cases a reflector 21 can be placed inside the housing such that light 17 emitted from the light source 20 is redirected out the aperture 14. Vertical cavity surface-emitting lasers (VCSELs) emit light perpendicular from the top surface, in contrast to conventional semiconductor lasers that emit light from the end of the device, so therefore would not need a reflector for light to be emitted out the aperture 14. LEDs emit light in all directions, and therefore would not need a reflector to aim light out the aperture 14 either. See FIG. 4.

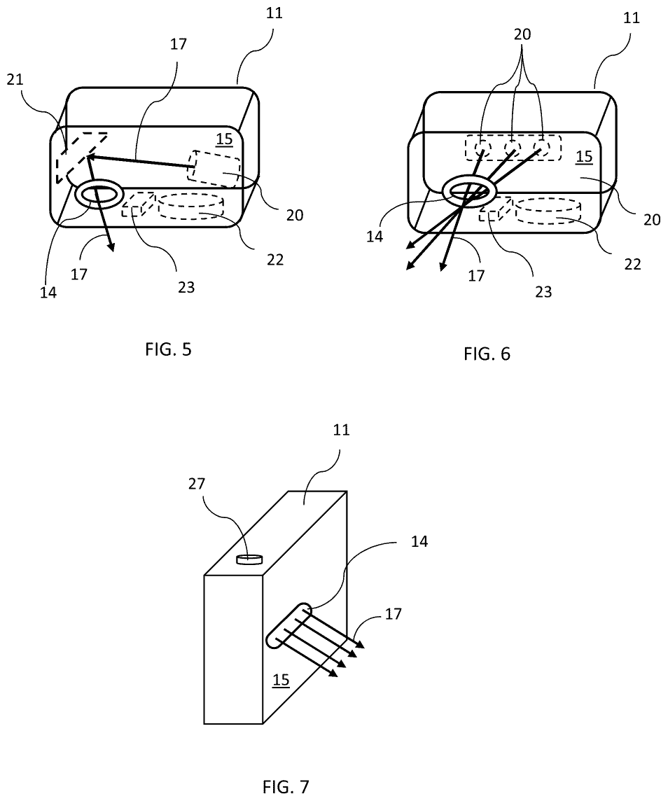

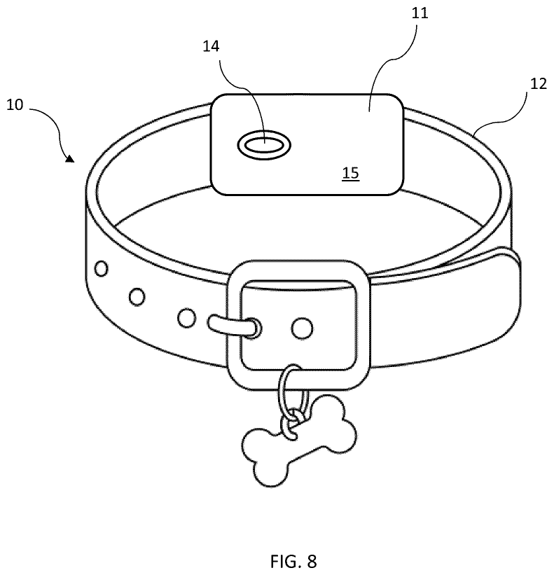

The aperture 14 can take any shape or size depending on the desired placement, size and shape of the light beam exiting the housing. FIGS. 5-6 show a small oval aperture 14. FIG. 7 shows a longer, rectangular aperture 14. With a larger aperture the exiting light can cover a larger portion of the animal's tissue, so that the light continues to impinge the desired treatment area in the event there is some shifting of the collar 10 during treatment. In some embodiments the apertures ring nearly the entire inside of the collar so that nearly the entire circumference of the neck is treated.

The light emitted from the aperture 14 is directed towards the tissue and typically is not visible to an observer when the device is in use. In some cases this is because the aperture abuts the animal's tissue and light cannot escape between the device and the tissue. In other cases the light is collimated, and the pathway the light travels so short, that the light beam doesn't spread outside the direct pathway to the tissue. Dense dark fur can also prevent light from escaping between the device and the tissue.

The applied light energy does not create heat, for example by using from conventional laser diode emitters of less than 1 W or from super pulse lights over 1 W. This low-level light therapy has an energy dose rate that causes no immediate or long-term detectable temperature rise of the treated tissue and no macroscopically visible changes in tissue structure. Consequently, the tissue impinged by the light is not heated and is not damaged. Because the tissue impinged by the light is not heated, no mechanism to cool the tissue is needed. The light is applied directly to the fur or tissue in the treatment area with no intervening temperature-reducing elements between the light-emitting device and the treatment area. The applied light energy may be applied continuously or applied with a pulse frequency or frequencies.

In some embodiments the emitted light has a specific shape. In a preferred embodiment a line of light is emitted from the device. Line-generating light therapy devices are known in the art, such as the ones disclosed in U.S. Pat. No. 6,746,473, which is incorporated herein by reference in its entirety. One embodiment includes a collimating lens and a line generating prism disposed in serial relation to the light energy source to receive and transform the generated beam of light energy into the line of light energy. In another embodiment a rod lens transforms the generated beam of light energy into the line of light energy. Alternatively, a suitable electrical or mechanical arrangement could be used to shape the light energy instead of optical elements. In the preferred embodiments, the emitted lines are parallel to each other, but alternatively the lines may be perpendicular or otherwise not parallel to each other. FIG. 7 illustrates a line of laser light emitted from the housing. In some embodiments light is emitted from non-coherent light sources, such as LEDs, and the light is emitted in multiple directions from the source. The wavelength of the light source is anywhere in the range of 400 nm to 1000 nm, depending on the desired treatment. In some embodiments only a single wavelength is used, for example 635 nm. Commercial semiconductor laser diodes have a spread of ±10 nm from nominal so, for a given desired wavelength, the light applied is within the spread from nominal. In other embodiments a range of wavelengths cover a desired color, for example red light.

Some methods described herein utilize light at defined ranges of wavelengths to target each complex of the electron transport chain at its peak absorption to increase the activity of each complex. This increases oxidative phosphorylation and mitochondrial function. Complex I and II are treated with violet-blue (400-490 nm) light. Complex III is treated with green (495-57 0nm) light. Complex IV is treated with red (620-700 nm) light. To achieve optimal oxidative phosphorylation all three colors are applied to the treatment area, simultaneously or non-simultaneously.

The three defined colors of light, violet-blue, green, and red, can be applied in several methods. One approach applies light energy of each of the three colors sequentially, activating one or two electron transport chain complexes with each color. Six sequences are available, each a method:

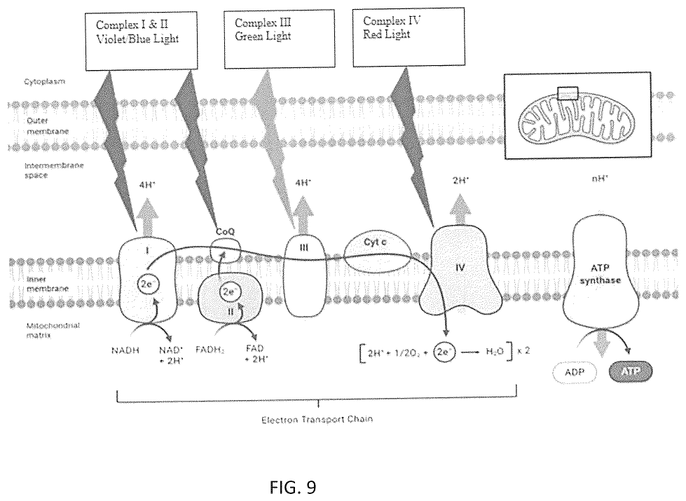

In one embodiment the activity of Complex I is increased by applying light energy having a violet-blue wavelength to a desired treatment area of an animal; the activity of Complex II is increased by applying light energy having a violet-blue wavelength to the treatment area; the activity of Complex III is increased by applying light energy having a green wavelength to the treatment area; and the activity of Complex IV is increased by applying light energy having a red wavelength to the treatment area. See FIG. 9.

In another embodiment the light energy is applied in order of green, violet-blue, and red, increasing the activity of Complex III, Complex I and Complex II, and Complex IV, respectively.

In another embodiment the light energy is applied in order of green, red and violet-blue, increasing the activity of Complex III, Complex IV, Complex I and Complex II, respectively.

In another embodiment the light energy is applied in order of red, violet-blue, and green, increasing the activity of Complex IV, Complex I and Complex II, and Complex III, respectively.

In another embodiment the light energy is applied in order of violet-blue, red, and green, increasing the activity of Complex I and Complex II, Complex IV, and Complex III, respectively.

In another embodiment the light energy is applied in order of red, green, and violet-blue, increasing the activity of Complex IV, Complex III, and Complex I and Complex II, respectively.

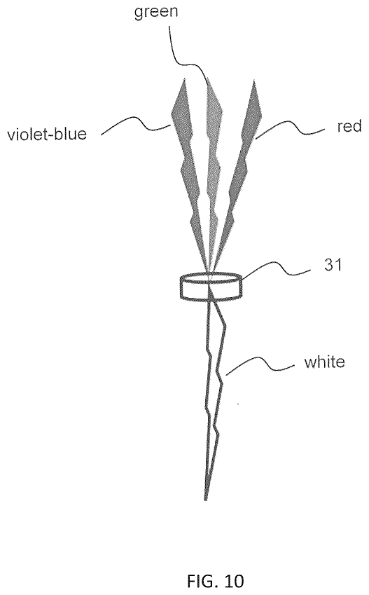

The light energy can be applied using a single light source that can emit all three wavelengths that impinge the animal at different locations so that all three colors can be seen on the tissue or fur at the same time. Alternatively, light energy can be applied using a single device that has a lens 31 such as a set of one or more prisms that all three colors pass through simultaneously, resulting in a white light on the tissue or fur of the animal. See FIG. 10. Instead of a single light source, light energy can be applied using multiple light source each of which emits a subset of the desired wavelengths. For example, two laser diodes may be used wherein the first diode emits red light and the second emits violet-blue and green.

The device is powered by a wireless power source 22, typically a battery. The battery may be rechargeable, by for example via USB or contact charging, or it may be non-rechargeable but replaceable, such as a button or coin battery. The device is controlled by an electronic controller 23 such as a microcontroller, which can be programmed with various pre-defined treatments. The device may be operated remotely using Wi-Fi or Bluetooth technology, such as from a mobile application on a smart phone. Optionally the device may have an electromechanical switch 27 on the housing to switch between pre-defined treatments as well as on/off. The device can be turned on periodically as a preventive treatment or more regularly for the healing of aliments.

The device can be used to maintain a health or cure a wide vary of mammalian system, such as integumentary system, skeletal system, muscular system, nervous system, endocrine system, cardiovascular system, lymphatic system, respiratory system, digestive system, urinary system, and reproductive system.

### Treatment Examples

A dog with lower limb arthritis may receive a light treatment with the phototherapy collar for 30 minutes per day. This is applied ongoing daily to provide relief and prevent symptoms from worsening.

A horse with a leg wound may receive three ten-minute light treatments daily with the phototherapy collar to promote wound healing and boost immunity to prevent infection.

A dog may receive a light treatment once a week with the phototherapy collar in order to maintain joint health.

A cat that undergoes neutering process may receive a five-minute light treatment four times a day with the phototherapy collar to assist with post-op pain and healing during the acute recovery phase.

A dog with renal disease may receive a forty-minute light treatment twice a day with the phototherapy collar.

A dog with phobias or anxiety may receive a five minute light treatment with the phototherapy collar, as needed to produce a calming effect.

An animal with cancer may receive 2-3 light treatments a day with the phototherapy collar until condition improves.

Livestock may receive daily light treatment with the photo therapy collar to increase fertility.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims and equivalents thereof.

1. A light therapy device for an animal having tissue, a neck, and a shoulder, the device comprising:
a. a housing comprising:
i. a tissue-facing surface;
ii. an aperture in the tissue-facing surface;
iii. at least one light source configured to emit light through the aperture;
iv. a power source; and
v. a controller;
wherein the housing is configured to be attached to a band or a harness, and the band or harness is configured to be secured to the animal such that the emitted light impinges the animal's tissue.

2. The light therapy device of claim 1 wherein the light is not visible to an observer when the device is in operation.

3. The light therapy device of claim 1 further comprising the band or the harness, wherein the band or the harness is secured around the animal's neck loosely enough that the housing falls naturally by gravity to the area where the animal's neck broadens out to the animal's shoulder.

4. The light therapy device of claim 1 wherein a dose rate of the emitted light causes no immediate or long-term detectable temperature rise of the animal's tissue and no macroscopically visible changes in tissue structure.

5. The light therapy device of claim 1 wherein the emitted light is red, green or violet-blue.

6. The light therapy device of claim 1 wherein the emitted light is red, green, and violet-blue.

7. A system for increasing mitochondrial membrane potential in an animal having tissue, a neck and a shoulder, the system comprising:
a. a light source configured to emit light through an aperture of a housing toward a first location, the housing configured to be secured at the first location proximate the animal's tissue proximate to a vein or artery, the vein or artery having mitochondria; and
b. a controller and a power source operably coupled to the light source, the controller configured to activate the light source such that the light traverses a pathway to arrive at and penetrate the animal's tissue proximate to a vein or artery, thereby increasing mitochondrial membrane potential in the mitochondria in the vein or the artery.

8. The system of claim 7 wherein a dose rate of the emitted light causes no immediate or long-term detectable temperature rise of the animal's tissue and no macroscopically visible changes in tissue structure.

9. The system of claim 7 wherein the light is not visible to an observer when the device is in operation.

10. The system of claim 7 wherein the emitted light is red, green or violet-blue.

11. The system of claim 7 wherein the emitted light is red, green, and violet-blue.

12. A method for increasing mitochondrial membrane potential in an animal having tissue, a neck and a shoulder, the method comprising:
a. applying one or more wavelengths of light to an animal's tissue proximate a vein or artery, the vein or artery having mitochondria.

13. The method of claim 12 wherein the applied light is emitted from a housing comprising:
i. a tissue-facing surface;
ii. an aperture in the tissue-facing surface;
iii. at least one light source configured to emit light through the aperture;
iv. a power source; and
v. a controller;
wherein the housing is configured to be attached to a band or a harness.

14. The method of claim 12 wherein the applied light is not visible to an observer.

15. The method of claim 12, wherein the band or the harness rests around the animal's neck loosely enough that the housing falls naturally by gravity to the area where the animal's neck broadens out to the animal's shoulder.

16. The method of claim 12 wherein the emitted light is red, green or violet-blue.

17. The method of claim 12 wherein the emitted light is red, green, and violet-blue.
